(19) United States
(12) Patent Application Publication
(10) Pub. No.: US 2008/0009676 A1
(43) Pub. Date: Jan. 10, 2008

(54) SURGICAL ENDOSCOPE FITTED WITH A FLEXIBLE BRANCHED TUBE

(75) Inventors: Thomas Wosnitza, Luneburg (DE); Uwe Scholer, Hoisdorf (DE)

Correspondence Address: RANKIN, HILL, PORTER & CLARK LLP, 38210 Glenn Avenue, WILLOUGHBY, OH, 44094-7808, US

(73) Assignee: OLYMPUS WINTER & IBE GMBH, Hamburg (DE)

(21) Appl. No.: 11/770,241

(22) Filed: Jun. 28, 2007

(30) Foreign Application Priority Data
Sep. 1, 2006 (DE) 10 2006 040 925.6
Jul. 5, 2006 (DE) 10 2006 031 415.8

(51) Int. Cl. A61B 1/005

(52) U.S. Cl. 600/143; 600/139

(57) ABSTRACT

A surgical endoscope (1) including a stem (2) and a manually bending branched tube (3, 17) issuing sideways from said stem wherein the branched tube (3, 17) consists of a single layer tube made of a material that can be durably deformed by hand.

### BACKGROUND OF THE INVENTION

The present invention relates to a surgical endoscope including a stem and a branched tube which branches sideways from the stem.

Medical endoscopes fitted with a laterally branched tube are used in many applications. Therein one operational duct runs within the stem as far as latter's proximal end whereas another operational duct deviates into a laterally branched tube. In many such endoscopes, the optics is housed in the straight operational duct and a surgical instrument, such as flexible pincers, a catheter or the like is housed within the laterally branched operational duct.

An endoscope of this kind is disclosed in the German patent document DE 103 51 185 B4. Therein, an operational duct runs in the straight stem made of a rigid material. The laterally branched tube houses a flexible fiber optics. The branched tube is rendered flexible by means of a very complex, two layer design comprising an elastically bending metal bellows on the inside and an elastic hose on the outside. The flexible branched tube of this design is made entirely of elastically resilient materials which, following the bending of the branched tube, would elastically return into their initial positions. To assure durable deformation, the branched tube comprises within it a complex spiral means itself made of an elastically resilient material.

The known, durably deformed branched tube offers the advantage that its free end may be moved into an arbitrary position, namely—in the known design—the advantage being to move the ocular mounted on the end of the branched tube into a convenient position of observation. The bending properties are adjusted in a manner that the branched tube is manually adjustable, i.e., that the surgeon may bend it into any desired shaped during surgery.

However, this known design also incurs the drawback of a multilayer construction and of the susceptibility of the outer elastic surface, because of inadequate sealing relative to the outside, allowing the penetration of moisture and water vapor at least into the metal bellows space allowed by this design of the state of the art. Such endoscopes are difficult to clean and disinfect.

The German patent document DE 197 56 629 A1 discloses a surgical endoscope fitted with a laterally branched tube subtending an angle to the endoscope stem. Using appropriate machinery, the branched tube may be manufactured to assume the desired shape, however, it is also rigid during normal surgery and cannot be manually adjusted to meet the particular needs of the surgeon.

The German patent document DE 39 23 851 C1 shows an endoscope used in nasal surgery and fitted with an obliquely branched tube connected by an adapter to another tube element which is bent but also rigid and which allows different orientations of its free terminal part by rotating the bent, rigid branched tube in its adapter.

### BRIEF SUMMARY OF THE INVENTION

The objective of the present invention is to improve the initial designs cited above with respect to manufacturing costs and ease of cleaning.

In the present invention, the branched tube consists of a single layer tube retaining its manually bent shape (unless and until being subsequently specifically treated into its initial, pre-bending state) and made of an appropriate material. Compared with the initially cited multilayer design of the state of the art, the single-layer design of the present invention offers substantial design simplicity, in particular, improving manufacturing costs and cleaning. The branched tube material easily deforms to allow manual deformation, illustratively even as regards a very short branched tube to be seized by the finger tips. During surgery, the surgeon is able to adapt the branched tube to his/her needs in the same manner as for the initially cited state of the art. Illustratively, the branched tube may be made of a durably deforming plastic or, it may be a suitably deforming metal such as soft iron. By means of its closed, smooth surface, the single layer tube is well sealing, easily cleaned and integral.

Advantageously, the branched tube shall be made of a memory alloy, namely an alloy substantially of nickel and titanium, which is both easily bent but, especially at higher temperatures, shall resume its original, un-bent shape. Such a return to the initial condition can be implemented in particular during the hot steam sterilization taking place at about 130° C. Following every use with any bending, the branched tube resumes its initial shape in the course of the hot steam sterilization ipso facto mandatory in the medical procedure.

The special-alloy branched tube segment may be made integrally with the endoscope stem, also made of the same material. Preferably, however, the branched tube is affixed by a junction site to the branched tube, as a result of which the stem per se may be made of a conventionally rigid, unbending material.

The joint defined, in particular may be a weld or a solder joint. However when joining rigid metal alloys to bending alloys and in particular to memory alloys—which typically are NiTi alloys—very considerable problems are encountered that in this instance preclude welding or soldering. A remedy is attained by making the tube parts of different materials and having the tube parts connected and sealed by a mechanical adapter. Preferably, such a tube adapter is designed to allow connecting to each other the tube ends without need for mechanical processing, such processing of memory alloys also being unusually difficult if the end part were to be a memory alloy or a plastic.

While the end part of a memory alloy can be durably deformed by bending, on the other hand the forces required for instance in view of the required diameters of an endoscope operational duct would be high. Advantageously therefore, the terminal element is designed as a bellows allowing for reducing the necessary bending forces.

Instead of making a terminal tube part from a memory alloy, said part advantageously may be in the form of a plastic tube reinforced by a memory wire sheath. As a result, the invention attains durable deformation provided by the memory wire and easier bending. The memory wire illustratively may be a wire mesh or a spiral hose.

The present invention is shown in illustrative and schematic manner in the appended drawings.

### BRIEF DESCRIPTION OF THE DRAWINGS

FIG. 1 is an endoscope sideview showing a lateral branched tube,

FIG. 2 is a cutaway of FIG. 1 showing possible bends of the branched tube,

FIG. 3 is a section along line 3-3 of FIG. 2 showing an elevation of other bend positions, and

FIG. 4 shows another embodiment mode fitted with a rotatable flange adapter.

### DETAILED DESCRIPTION OF THE INVENTION

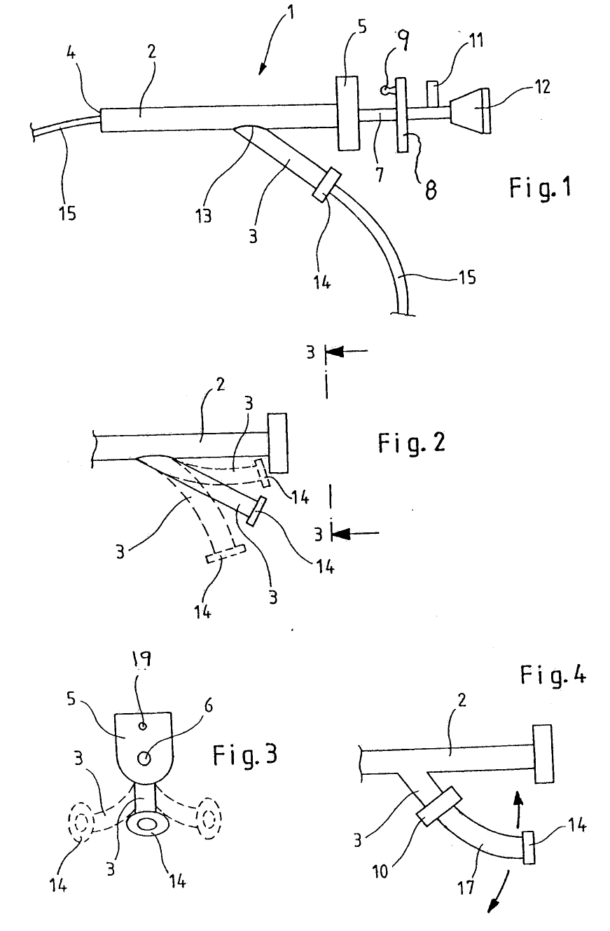

FIG. 1 shows a highly schematic, simple embodiment of a surgical endoscope 1 illustratively used on the human bladder. The endoscope 1 comprises an elongated stem 2 made of a rigid metal fitted with lateral branched tube 3 subtending an angle to the axis of said stem 1.

The distal end 4 of the stem 2 is open whereas an adapter 5 is configured at its proximal end through which an optics 7 may be inserted into a hole 6 (FIG. 3). Said optics 7 is fitted with a mating part 8 which upon insertion can be locked by means of a pin 9 into a hole 19 of the adapter 5. The optics 7 also is fitted at its proximal end with a lateral outlet stub 11 to connect a fiber optics cable, further with an ocular 12.

The branched tube 3 is made of another material than the stem 2 and its junction to this stem is a seam 13. At its free end the branched tube 3 comprises an insertion element 14 through which may be inserted, as indicated, a flexible surgical implement 15, for instance flexible pincers, a laser fiber or a catheter.

In its position indicated in FIG. 1, the branched tube 3 is configured laterally and obliquely at a predetermined angle to the stem 2 and allows inserting the surgical implement 15 in the shown position. This position may be inconvenient for some procedures and illustratively it may then be more convenient to insert the surgical implement 15 parallel to the optics 7.

For that purpose, the branched tube 3 is made of a plastic or preferably a metal flexible tube that is durably deforming, preferably plastically, and which can be deformed into arbitrary angular positions as shown in FIG. 3. The bending parameter shall be adjusted in a manner that even under difficult circumstances, bending still shall be feasible even if only carried out with the finger tips while the shape shall be retained at typical surgery loads. In this manner and following appropriate bending of the branched tube 3, the surgical implement 15 still may be inserted, for instance parallel to the optics 7 or perpendicularly to it, into the insertion part 14 of the branched tube 3. FIG. 2 shows how the branched tube 3 may be bent to different extents in the plane (of the drawing) subtended by the stem 2 and the branched tube 3.

FIG. 3 shows in axial elevation that the branched tube 3 also may be bent sideways, that is, out of the plane of the drawing of FIG. 2.

The branched tube 3 of the embodiment mode of FIGS. 1 through 3 illustratively may be made of durably bending soft iron allowing easy cleaning of its smooth surface and being easily bent into an arbitrary shape, said branched tube being joined for instance by laser welding illustratively at the seam 13 to the stem 2 which is made of another material.

The material of the branched tube 3 is selected to be bendable easily enough that the branched tube 3 can be bent manually in any desired shape as shown in FIGS. 2 and 3. As a result, the surgeon in the operating room always can meet the requirements of the moment.

In the embodiment mode of FIGS. 1-3, the special-material branched tube 3 also may be joined in other ways to the conventional-material stem 2, for instance using a flange joint as shown in FIG. 4. The materials of the stem 2 and the branched tube 3 also may be the same, provided both allow satisfactory manufacture. However, in the typical case, the stem 2 is made of a rigid material, and separate manufactures will be required.

FIG. 4 shows another embodiment mode which retains the previous references where possible.

In this embodiment, the branched tube 3 is short, straight and may be made of the same material as the stem 2. At its free end, the branch tube 3 is fitted with a flange 10 receiving a rigid tube element 17 bent into a specified curvature. Just like the branched tube 3 of FIGS. 1-3, the tube element 17 is made of a manually bendable material, for instance a flexible metal, a plastic or the like.

By means of the deforming branched tube 3 respectively tube element 17, an operational implement may be repositioned, and it also may receive a flexible optics or an optic fiber or serve as a rinsing duct.

In the embodiment mode discussed in relation to FIGS. 1 through 3, the stem 2 of the endoscope 1 may be made of an appropriate rigid material such as high grade or stainless steel. The branched tube 3, on the other hand, is made of another material which is durably deformed by bending as indicated in FIGS. 2 and 3. The latter material illustratively may be soft iron joined, for instance, by welding by the seam 13 to the stem 2.

However, such material mating already raises junction problems. Memory alloys, which typically are NiTi alloys, on account of their good deformation properties for the branched tube 3, are much more suitable. On the other hand, memory alloys entirely preclude being welded or soldered at the seam 13 to the metal of the stem 2. Accordingly, when using other alloys and in particular memory alloys for the branched tube 3, a design is used which is described in relation to FIG. 4 and denoted by the related references.

The branched tube of FIG. 4 may comprise an initial segment 3, for instance, made of the same material as the stem 2 and easily joined to it. In one embodiment mode, a separate terminal element 13 of the branched tube consists of a suitably bending tube, in particular made of a memory alloy or illustratively also of plastic. The branched tube parts 3, 17 prohibit being welded to each other, they are joined instead by a tube adapter 10 which in particular is designed in a manner that the terminal element 17 may be cut off just at its tip near the tube adapter 10 and therefore need not be processed for purposes of tube coupling.

Appropriate tube adapters are known, for instance, from plumbing techniques to connect water pipes to water sinks. They are fitted with sealing tightening screws and may include a pipe enclosing the two branched tube elements 3, 17, assuring reliable force transmission between these elements 3, 17. When a substantial force is applied to bend the terminal element 17.

In such an embodiment the terminal element 17 alone may be deformed and may be bent arbitrarily, for instance, as indicated in FIG. 2, or by sideways bending, as shown in FIG. 3.

A flat memory-alloy tube as shown by the terminal element 17 in FIG. 4 will cause considerably difficulties if the attempt is made to bend it in the shown range of diameters. The memory-alloy terminal element 17 therefore can be designed in an embodiment not illustrated, in the form of a bellows that would result in easier bending.

In another, omitted embodiment mode, the terminal element 17 also may be a plastic hose reinforced (armored) in by a memory-alloy wire, said wire illustratively being helical or mesh-wise. The elastically resilient plastic is kept, after being bent by the durably deforming memory wire, in its new shape.

When using memory-material for the bending branched tube, said memory material, for instance an NiTi alloy, may be adjusted in such manner that at higher temperature it shall return to its initial, bent shape. This restoring temperature may be selected to be that which is reached at the endoscope in the autoclaving procedure required for sterilization, that is a temperature of about 130° C. Thereupon the endoscope 1 may be used for surgery when bending the branched tube is required. Upon completion of surgery and during autoclaving, the branched tube is restored to its initial shape and then may be bent into another one at the next surgery.

1. A surgical endoscope (1) comprising:
a stem (2), and
a branched tube (3, 17) which branches sideways from said stem (2) and which can be bent manually,
wherein the branched tube (3, 17) consists of a single-layer tube made from a material that can be durably bent by hand.

2. The endoscope as claimed in claim 1, wherein the bending branched tube (3, 17) is made of metal which can be durably bent.

3. The endoscope as claimed in claim 2, wherein the bending branched tube (3, 17) consists of a memory alloy and is restored at higher temperature to its initial shape.

4. The endoscope as claimed in claim 1, wherein the bending branched tube (3, 17) is affixed to the stem

5. The endoscope as claimed in claim 4, wherein the junction is implemented by a tube adapter (10).

6. The endoscope as claimed in claim 1, wherein the bending branched tube (3, 17) is a bellows.

7. The endoscope as claimed in claim 1, wherein the bending branched tube (3, 17) is a plastic hose armored in memory-alloy wires.
